(19) United States
(12) Patent Application Publication
(10) Pub. No.: US 2024/0221862 A1
(43) Pub. Date: Jul. 4, 2024

(54) SYSTEM AND METHOD FOR ELECTRONIC IDENTIFICATION OF BIOMARKERS ASSOCIATED WITH PATHOLOGY

(71) Applicant: Innoplexus AG, Eschborn (DE)

(72) Inventors: Irfan Tamboli, Pune (IN); Vinay Chandil, Gwalior (IN); Harsh Sharma, Indore (IN); Om Sharma, Pimpri-Chinchwad (IN); Ruchika Sharma, Lucknow (IN)

(73) Assignee: Innoplexus AG, Eschborn (DE)

(21) Appl. No.: 18/148,535

(22) Filed: Dec. 30, 2022

(51) Int. Cl. G16B 5/00; G06N 3/0455; G16B 25/00; G16B 40/20

(52) U.S. Cl. CPC G16B 5/00; G06N 3/0455; G16B 25/00; G16B 40/20

(57) ABSTRACT

A system for electronic identification of one or more biomarkers associated with a pathology. The system includes a processor configured to receive an expression data and utilize a predefined degenerative model to generate a Transcription Factor (TF)-target interaction information based on the received expression data. The processor is configured to execute the predefined degenerative model to generate a Gene Regulatory Network (GRN) based on the TF-target interaction information and construct a hierarchical network defining hierarchical relationships among two or more nodes and one or more edges of the GRN. The processor is configured to prioritize a set of TFs and a set of targets using a Protein-Protein Interaction (PPI) network and construct a modified GRN. The system efficiently and reliably identifies the one or more biomarkers associated with the pathology by use of a combination of the GRN, the hierarchical network and the PPI network.

### TECHNICAL FIELD

The aspects of the disclosed embodiments relate generally to the field of identification of biomarkers; and more specifically, to a system and a method for electronic identification of one or more biomarkers associated with a pathology.

### BACKGROUND

Conventionally, the process of biomarker identification has followed a gene-centric approach in which, either a specific gene is analysed based on previous biological assumptions or a gene(s) is selected based on a differential behavior without connection to an upstream and a downstream molecular mechanism. Such process is partially effective due to lack of segregation of a single gene or a group of genes between different data types.

Currently, certain attempts have been made to identify the biomarkers, such as use of conventional Gene Regulatory Networks (GRNs). Many dynamic processes in a cell and organisms, such as cell cycle, differentiation, development, cell division are governed by the conventional GRNs. Thereafter, several GRN inference methods are developed that use an expression data of different physiological conditions, such as diseased vs healthy to deduce interactions between Transcription Factors (TFs) and targets. However, the conventional GRN algorithms are inadequate due to high noise in the expression data that contributes to a high number of false positives. Moreover, many GRN algorithms rely only on the conventional GRN that investigates and takes into account TF-target interactions at gene level and leaves out the interactions at protein level. Additionally, the variety of algorithms, such as regression, correlation, Bayesian (e.g., simple dynamic), information theory, phixer and the like, are evaluated for their utility to infer the conventional GRNs. Algorithms, such as Linear Profile Likelihood (LiPLike), Priori-Fused Boosting Network inference method (PFBNet) etc., are used to reduce high dimensionality and noise in the expression data. Such noise reduction algorithms mostly relied only on modification without taking into account any Protein-Protein Interaction (PPI) or biological data. As a reference database, many of aforementioned algorithms make use of single databases, few of which have experimentally validated TF-target interactions and few ones lack other required attributes about the TF-target interactions. The algorithms which rely on gene-based stratification make use of differential expression data of individual genes. Such a simplistic view of pathology ignores interactions of multiple genes that work together to form complex cellular networks and evoke lower confidence. Thus, there exists a technical problem of how to efficiently and reliably identify one or more biomarkers associated with a pathology and thereby, developing novel therapeutic strategies and generating new avenues for targeting relevant phenotypes and diseases.

Therefore, in light of the foregoing discussion, there exists a need to overcome the aforementioned drawbacks associated with the conventional methods of identification of one or more biomarkers that drive associated transcriptional changes.

### SUMMARY

The aspects of the disclosed embodiments are directed to a system and a method for electronic identification of one or more biomarkers associated with a pathology. An aim of the disclosed embodiments is to provide an improved system and an improved method for electronic identification of one or more biomarkers associated with a pathology.

One or more advantages of the disclosed embodiments are achieved by the solutions provided in the enclosed independent claims. Advantageous implementations of the present disclosure are further defined in the dependent claims.

In an aspect, the aspects of the disclosed embodiments provide a system for electronic identification of one or more biomarkers associated with a pathology. The system comprises a processor configured to receive an expression data comprising a plurality of datasets related to different physiological conditions of a plurality of subjects and utilize a predefined degenerative model to generate a Transcription Factor (TF)-target interaction information based on the received expression data. The processor is further configured to execute the predefined degenerative model to generate a Gene Regulatory Network (GRN) based on the TF-target interaction information, where the GRN comprises a plurality of TFs and a plurality of targets as two or more nodes, and a plurality of relationships among the plurality of TFs and the plurality of targets as one or more edges. The processor is further configured to construct a hierarchical network defining hierarchical relationships among the two or more nodes and the one or more edges of the GRN and prioritize a set of TFs and a set of targets among the plurality of TFs and the plurality of targets, respectively, using a Protein-Protein Interaction (PPI) network. The processor is further configured to construct a modified GRN based on the prioritized set of TFs and the prioritized set of targets to identify the one or more biomarkers associated with the pathology.

The disclosed system efficiently and reliably identifies the one or more biomarkers associated with the pathology by use of a combination of the GRN, the hierarchical network and the PPI network. The reliable identification of the one or more biomarkers supports development of novel therapeutic strategies. The hierarchical network is based on hierarchy of TFs and targets which makes easy to understand direct and indirect spatial interactions between TFs and targets. Moreover, the hierarchical network includes the identification of master regulators or top TFs (i.e., highly active TFs), and top targets (i.e., highly regulated genes), which is further utilized to generate the modified GRN. The modified GRN provides TF-target interactions not only at the gene level but also, at the protein level. Moreover, the system provides a comprehensive and accurate understanding of gene expression regulation and dysregulation at transcriptional level that takes into account the translational or protein level interactions. In addition to identification of the dysregulated genes, the system is focused on identification of TEs which are involved in gene expression and thereby may be associated with the disease. In addition to prioritization of the TFs and targets, the clusters of genes and TFs are identified which may be specifically highlighted for a particular disease. For example, the dysregulation of transcription in cancer makes use of the modified GRN very useful when it comes to cancer diagnostics and novel therapeutics.

In another aspect, the aspects of the disclosed embodiments provide a method of electronic identification of one or more biomarkers associated with a pathology. The method comprises receiving, by a processor, an expression data comprising a plurality of datasets related to different physiological conditions of a plurality of subjects and utilizing, by the processor, a predefined degenerative model to generate a Transcription Factor (TF)-target interaction information based on the received expression data. The method further comprises executing, by the processor, the predefined degenerative model to generate a Gene Regulatory Network (GRN) based on the TF-target interaction information, where the GRN comprises a plurality of TFs and a plurality of targets as two or more nodes, and a plurality of relationships among the plurality of TFs and the plurality of targets as one or more edges. The method further comprises constructing, by the processor, a hierarchical network defining hierarchical relationships among the two or more nodes and the one or more edges of the GRN and prioritizing, by the processor, a set of TFs and a set of targets among the plurality of TFs and the plurality of targets, respectively, using a Protein-Protein Interaction (PPI) network. The method further comprises constructing, by the processor, a modified GRN based on the prioritized set of TFs and the prioritized set of targets to identify the one or more biomarkers associated with the pathology

The method achieves all the advantages and technical effects of the disclosed system of the present disclosure.

It is to be appreciated that all the aforementioned implementation forms can be combined. It has to be noted that all devices, elements, circuitry, units and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of a system for electronic identification of one or more biomarkers associated with a pathology, in accordance with an embodiment of the present disclosure;

FIGS. 2A to 2C collectively is a flowchart of a method of electronic identification of one or more biomarkers associated with a pathology, in accordance with an embodiment of the present disclosure;

FIG. 3A is a diagram of a Gene Regulatory Network (GRN), in accordance with an embodiment of the present disclosure;

FIG. 3B is a diagram of a hierarchical network, in accordance with an embodiment of the present disclosure;

FIGS. 4A-4B collectively represent conversion of a random GRN to a layered GRN, in accordance with an embodiment of the present disclosure;

FIG. 5 is a diagram of superimposition of a GRN, a hierarchical network and a Protein-Protein Interaction (PPI) network, in accordance with an embodiment of the present disclosure;

FIG. 6 illustrates a Deep Structural Equation Modelling (DeepSEM model), in accordance with an embodiment of the present disclosure; and

FIG. 7 is a flowchart that describes how hierarchical scores are assigned to each of TFs and targets in a hierarchical network, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

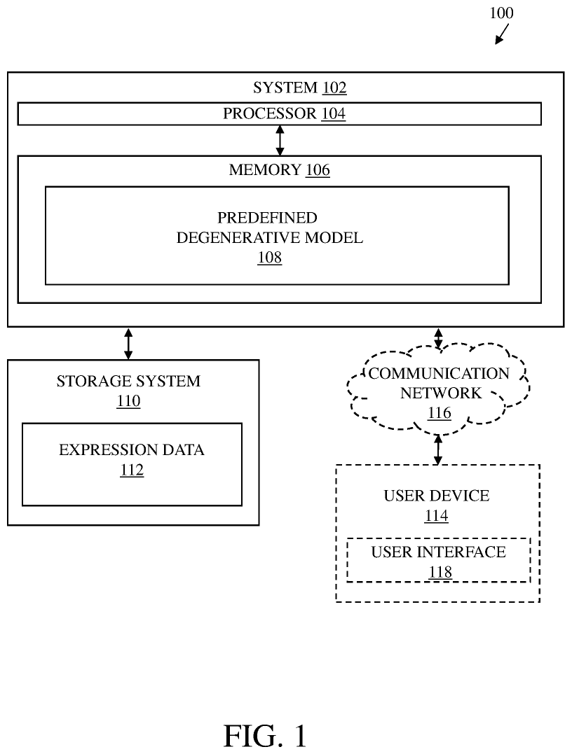

FIG. 1 is a block diagram of a system for electronic identification of one or more biomarkers associated with a pathology, in accordance with an embodiment of the present disclosure. With reference to FIG. 1, there is shown a block diagram 100 of a system 102 that includes a processor 104 communicably coupled to a memory 106. The memory 106 includes a predefined degenerative model 108. The memory 106 is further connected to a storage system 110 configured to store an expression data 112. Optionally, the system 102 is connected to a user device 114 through a communication network 116. The user device 114 comprises a user interface 118.

The system 102 is used for electronic identification of one or more biomarkers associated with a pathology. Conventionally, the one or more biomarkers identification relies on a gene-centric approach that uses differential expression data of individual genes. Such a simplistic view of pathology ignores interaction of multiple genes that work together to form complex cellular networks. Single gene biomarkers do not consider any clustering of genes falling into one category and prioritization of Transcription Factors (TFs)-targets interactions and therefore, less effective for biomarkers identification. In the present disclosure, instead of the single gene, clusters of genes and TFs are considered for reliable identification of the one or more biomarkers associated with a pathology. In case of any disease or a given condition, many genes are dysregulated, either upregulated or downregulated, which contribute to the disease. So, in addition to identification of the dysregulated genes, the present disclosure is focused on identification of TFs which are involved in gene regulation and thereby may be associated with the disease. Moreover, the present disclosure identifies the relationship between TFs and targets, and prioritization of the TFs-targets interactions. Moreover, the present disclosure is focused on identification of clusters of TFs and targets which should be specifically highlighted for a particular disease. After identification of TFs-targets interactions and the major clusters of targets, the biomarkers are accurately identified based on TFs-target interactions and the clusters resulting in development of novel therapeutic strategies and generation of new avenues for targeting relevant phenotypes and diseases.

The processor 104 may include suitable logic, circuitry, and/or interfaces that is configured to respond and process the instructions required to drive the system 102. Furthermore, the processor 104 may refer to one or more individual processors, processing devices and various elements associated with a processing device that may be shared by other processing devices. Additionally, the one or more individual processors, processing devices and elements are arranged in various architectures for responding to and processing the instructions to drive the system 102. In an implementation, the processor 104 may be an independent unit and located outside the system 102. Examples of the processor 104 may include, but are not limited to a hardware processor, a digital signal processor (DSP), a microprocessor, a microcontroller, a complex instruction set computing (CISC) processor, an application-specific integrated circuit (ASIC) processor, a reduced instruction set (RISC) processor, a very long instruction word (VLIW) processor, a state machine, a data processing unit, a graphics processing unit (GPU), and other processors or control circuitry.

The memory 106 may include suitable logic, circuitry, and/or interfaces that is configured to store data and the instructions executable by the processor 104. Examples of implementation of the memory 106 may include, but are not limited to, an Electrically Erasable Programmable Read-Only Memory (EEPROM), Random Access Memory (RAM), Read Only Memory (ROM), Hard Disk Drive (HDD), Flash memory, Solid-State Drive (SSD), or CPU cache memory. The memory 106 may store an operating system or other program products (including one or more operation algorithms) to operate the system 102. In an embodiment, the memory 106 may be configured to store the predefined degenerative model 108. Furthermore, the memory 106 is connected to the storage system 110 to receive the expression data 112 which comprises a plurality of datasets related to different physiological conditions of a plurality of subjects. The plurality of subjects includes a number of patients, some of which are drug-treated for a disease cure and some patients are non-treated. Therefore, the plurality of datasets includes data of the patients which are drug-treated as well as data of the patients which are non-treated.

The storage system 110 may include suitable logic, circuitry, and/or interfaces that is configured to store the expression data 112. Examples of implementation of the storage system 110 are similar to examples of implementation of the memory 106. In an embodiment, the storage system 110 may be a database system that may be configured to store the expression data 112 comprising the plurality of datasets related to different physiological conditions of the plurality of subjects.

The communication network 116 may include suitable logic, circuitry, and/or interfaces through which the system 102 is connected to the user device 114. Examples of implementation of the communication network 116 may include, but are not limited to, a cellular network (e.g., a 2G, a 3G, long-term evolution (LTE) 4G, a 5G, or 5G NR network, such as sub 6 GHZ, cmWave, or mmWave communication network), a wireless sensor network (WSN), a cloud network, a Local Area Network (LAN), a vehicle-to-network (V2N) network, a Metropolitan Area Network (MAN), and/or the Internet.

The user device 114 may include suitable logic, circuitry, and/or interfaces that is used by a user (not shown in FIG. 1) for analyzing the identified one or more biomarkers associated with the pathology. The user device 114 comprises the user interface 118 which displays the identified one or more biomarkers associated with the pathology to the user. Examples of implementation of the user device 114 may include, but are not limited to, a computer, mobile phone, laptop, a display device, and the like.

In operation, the aspects of the disclosed embodiments provide the system 102 for electronic identification of one or more biomarkers associated with a pathology. The system 102 comprises the processor 104 configured to receive the expression data 112 comprising the plurality of datasets related to different physiological conditions of the plurality of subjects. The plurality of subjects corresponds to the plurality of patients and the plurality of datasets corresponds to the datasets captured in different physiological conditions of the plurality of patients. For example, in an implementation, the plurality of datasets may include the data of some patients which are kept under a disease control as well as the data of the patients which suffer from a disease. In another implementation, the plurality of datasets may include the data of some patients which are drug treated for a disease cure and the data of the patients which are non-treated. In a yet another implementation, the plurality of datasets may include the data of some patients which are suffering from the disease and the data of a number of healthy persons.

In accordance with an embodiment, the expression data 112 comprises either high throughput sequencing (HTS) data, or a micro-array data or a single cell sorting, RNA extraction, reverse transcription, amplification, library construction, sequencing and subsequent bioinformatic analysis (scRNA-seq) data. In an implementation, the expression data 112 may include the HTS data. Generally, the HTS is a drug discovery process that allows an automated testing of large numbers of chemical and/or biological compounds for a specific biological target. In another implementation, the expression data 112 may include the micro-array data. The micro-array data is a tool used to detect the expression of thousands of genes simultaneously. In a yet another implementation, the expression data 112 may include the scRNA-seq data. The scRNA-seq measures the Ribonucleic Acid (RNA) molecules within each cell of a given sample. Additionally, the expression data 112 may include data of Differentially Expressed Genes (DEG).

The processor 104 is further configured to utilize the predefined degenerative model 108 to generate a Transcription Factor (TF)-target interaction information based on the expression data 112. The predefined degenerative model 108 is used to generate causal relationship between TF and targets with high certainty using the expression data 112. Moreover, the predefined degenerative model 108 is configured to use Structural Equation Modelling (SEM) to determine multivariate causal relationships between TF and target. Generally, a TF is defined as a protein, which is a gene product and regulate expression of a lot of genes by binding to a gene promotor. In Deoxyribonucleic Acid (DNA), multiple genes are there, and each gene has a promotor in the beginning which can either switch on or off the expression of that particular gene. In the present disclosure, the target corresponds to a gene. Therefore, it may be stated that, the predefined degenerative model 108 is used to generate relationship between TF and genes by use of the expression data 112.

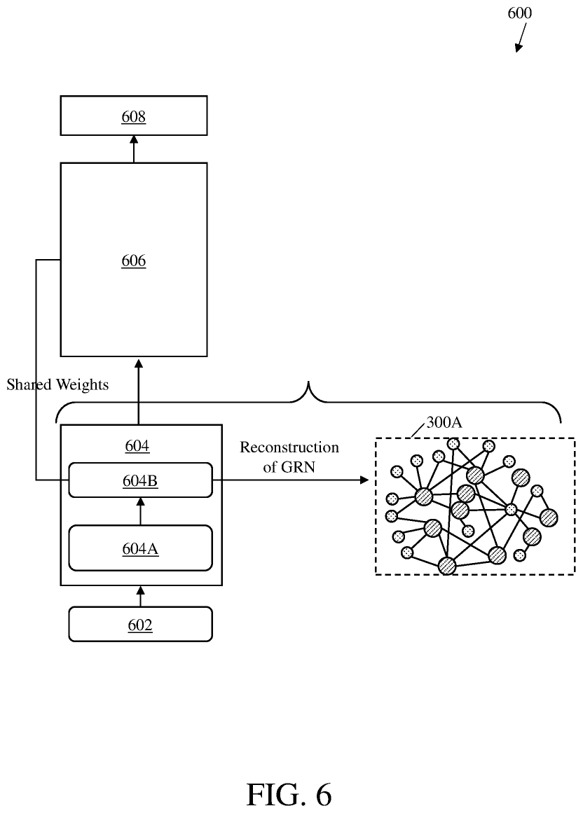

In accordance with an embodiment, the predefined degenerative model 108 is an autoencoder model. The predefined degenerative model 108 may also be referred to as either a TF Express model or DeepSEM model, described in detail, for example, in FIG. 6. The DeepSEM model is an autoencoder model. The DeepSEM model is a neural network version of a conventional SEM model to explicitly model the relationships among genes.

In accordance with an embodiment, the generation of the TF-target interaction information comprises normalization of a plurality of databases resulting to a normalized database. The TF-target interaction information is generated using different databases. Only those databases are considered which have relevant and experimentally validated data. Examples of the databases may include, but are not limited to, hTFtarget database, TcoF-DB v2, TFacts, TF2DNA, ENCODE, and the like. Each of aforementioned databases has information about the TFs and targets. Every database has a different number of TF-target relationships. The common factor among aforementioned databases is Uniprot ID. Few databases have the information of mode of regulation while another database has the information about the tissue in which a particular connection exists. Since each database is unique in its own way therefore, each of aforementioned databases is stored at one place in such a manner that for each gene, information from each database can be collected. Therefore, each of aforementioned databases is normalized to generate the normalized database. The normalized database may be stored in form of a dictionary, where key is the attribute and values are the corresponding information. In an implementation, the normalized database may be of a size 1.47 GB which may be stored in an Elasticsearch database or a Mongo database. The normalized database has all the relevant information required for TF-target interactions, resulting in time saving and an increased efficiency. Moreover, the normalized database may have 21971242 TF-target interactions. The normalized database may also be referred to as a TFT-database which describes TF-target interactions by considering experimentally validated data across various databases.

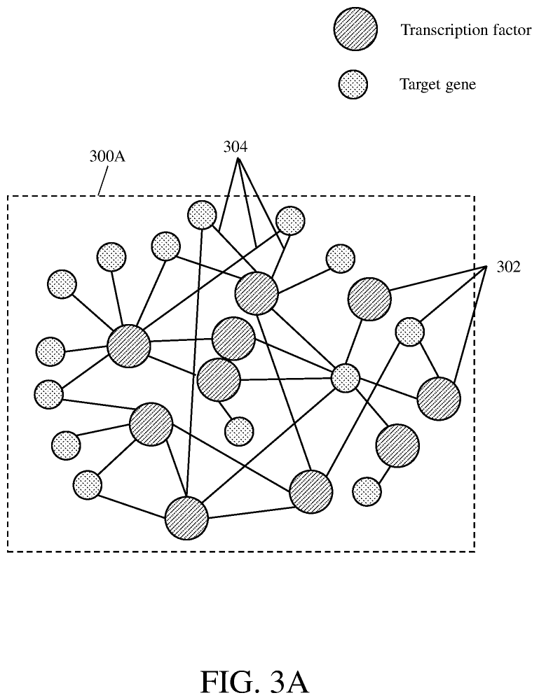

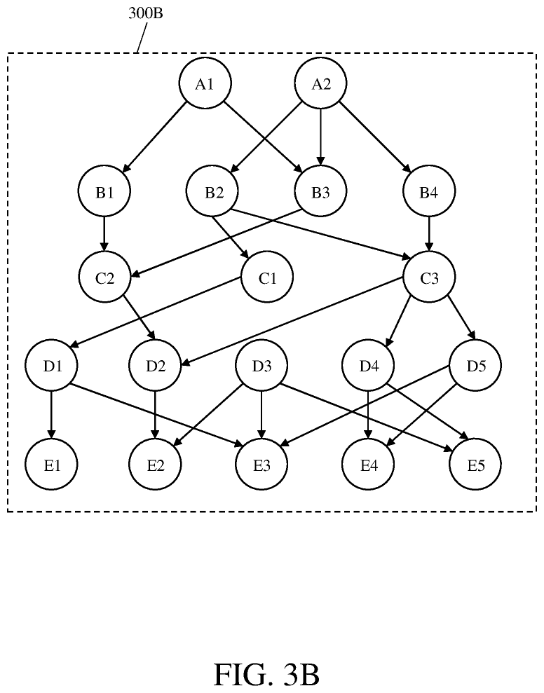

The processor 104 is further configured to execute the predefined degenerative model 108 to generate a Gene Regulatory Network (GRN) based on the TF-target interaction information, wherein the GRN comprises a plurality of TFs and a plurality of targets as two or more nodes, and a plurality of relationships among the plurality of TFs and the plurality of targets as one or more edges. The predefined degenerative model 108 is further used to generate the GRN based on the causal relationship between TF and targets determined using the expression data 112. The causal relationship between TF and targets may be termed as weighted relationships between TFs and targets. The weights (may also be represented as W) merely represent the connections between TFs and targets. The absolute value of each element of W is used to rank the possibility of the regulatory relationships between genes. The GRN provides mechanistic interactions between TFs and its targets. The nodes in the GRN represent the plurality of TFs and the plurality of targets (i.e., genes) and edges represent the relationship between the plurality of TFs and the plurality of targets. The generated GRN may also be referred to as a random GRN. An exemplary scenario of the GRN is described in detail, for example, in FIG. 3A.

The processor 104 is further configured to construct a hierarchical network defining hierarchical relationships among the two or more nodes and the one or more edges of the GRN. The generated weighted relationships of TFs-targets are used as an input for generation of the hierarchical network that defines the hierarchical relationships between the plurality of TFs and the plurality of targets. An exemplary scenario of the hierarchical network is described in detail, for example, in FIG. 3B.

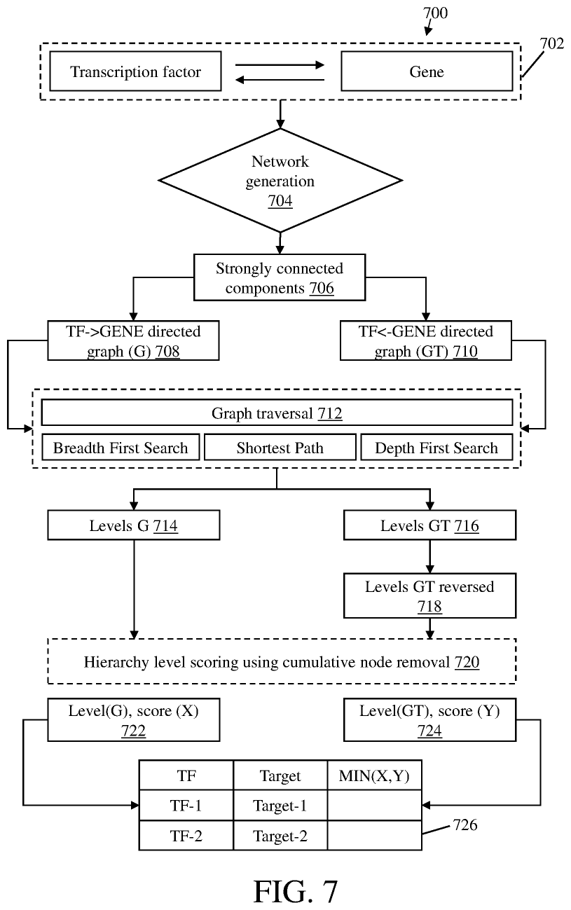

In accordance with an embodiment, the processor 104 is further configured to execute a Strongly Connected Components (SCC) operation in the hierarchical network to generate one or more directed acyclic graphs. The processor 104 is further configured to traverse the generated one or more directed acyclic graphs using one of: a Breadth First Search (BFS) operation, a Shortest Path (SP) operation and a Depth First Search (DFS) operation to determine distance among the two or more nodes of the GRN and assign a hierarchical score to each of the two or more nodes of the GRN using a cumulative node removal technique to quantify a degree of hierarchy in the GRN. The processor 104 is further configured to reduce the number of nodes (i.e., the plurality of TFs and the plurality of targets) in the hierarchical network by executing the SCC operation. The SCC operation is further executed for generation of the one or more directed acyclic graphs of TFs and targets. Generally, the SCC operation represents a graph where there is a path between each of vertex. Alternatively stated, each TF is reachable from every other TF and target. The generated one or more directed acyclic graphs of TFs and targets are traversed using one of the BFS operation, the SP operation and the DFS operation to determine the distance of the nodes from an input gene (or a protein) for which it is required to find stable and deterministic connections. The traversing of the one or more directed acyclic graphs of TFs and targets is performed to optimize and analyse the hierarchical network, described in detail, for example, in FIG. 7. Furthermore, each node of the GRN is assigned the hierarchical score (HS) to quantify the degree of hierarchy in the hierarchical network.

In accordance with an embodiment, the processor 104 is further configured to identify one or more master regulator nodes among the two or more nodes of the GRN based on the assigned hierarchical score to each of the two or more nodes of the GRN. The hierarchical score assigned to each node of the GRN is used for identification of the one or more master regulator nodes in the GRN. The one or more master regulator nodes correspond to a subset of the nodes of the GRN representing the plurality of TFs.

In accordance with an embodiment, the processor 104 is further configured to identify one or more regulated or dysregulated targets among the two or more nodes of the GRN based on the assigned hierarchical score to each of the two or more nodes of the GRN. The hierarchical score assigned to each node of the GRN is used for identification of highly regulated or dysregulated genes. The one or more regulated or dysregulated genes (or targets) correspond to a subset of nodes of the GRN representing the plurality of targets.

In accordance with an embodiment, the processor 104 is further configured to identify one or more clusters of the plurality of targets, wherein each cluster belongs to a specific data type and correlate the identified one or more clusters of the plurality of targets with one or more of the plurality of TFs in the GRN. The identified one or more clusters of the plurality of targets corresponds to clusters of genes where each cluster has a specific property. The identified clusters of genes are further correlated with the plurality of TFs which may be arranged either in a horizontal or a vertical manner in the GRN. Moreover, the identified clusters of genes and TFs may be specifically highlighted for a particular disease.

The processor 104 is further configured to prioritize a set of TFs and a set of targets among the plurality of TFs and the plurality of targets, respectively, using a Protein-Protein Interaction (PPI) network. The PPI network is constructed using all the entities of the GRN and PPI network parameters are used to further prioritize TF-target interactions and reduce noise. The PPI network provides biological insights or functional implications of which genes are differentially expressed and how the protein-protein interactions are enriched. Using the protein-protein interactions, most highly connected proteins can be identified. The most highly connected proteins correspond to the set of TFs and the set of targets among the plurality of TFs and the plurality of targets which are prioritized. Additionally, the PPI network makes use of a normalized data from publicly available databases and literature. Conventionally, the PPI network is used independently to provide the biological insights, which lacks reliability. In the present disclosure, the PPI network is used in combination with the GRN and the hierarchical network therefore, the biological insights obtained by use of the PPI network are more reliable.

The processor 104 is further configured to construct a modified GRN based on the prioritized set of TFs and the prioritized set of targets to identify the one or more biomarkers associated with the pathology. The GRN, the hierarchical network and the PPI network, all three networks and other biomarker feasibility parameters are used to identify the one or more biomarkers associated with the pathology. The modified GRN provides more reliable mechanistic interactions between TFs and targets, which further benefits the process of drug design and discovery. The transcriptional regulation is a fundamental cellular regulatory mechanism which is associated with physiological as well as pathological changes in a system therefore, gaining improved mechanistic interactions between TFs and targets by use of the modified GRN has a high relevance to many different aspects of disease biology and drug discovery.

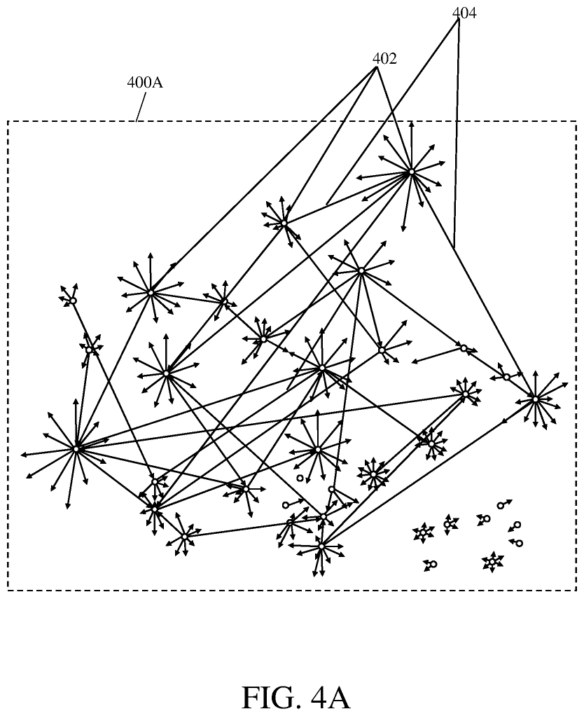

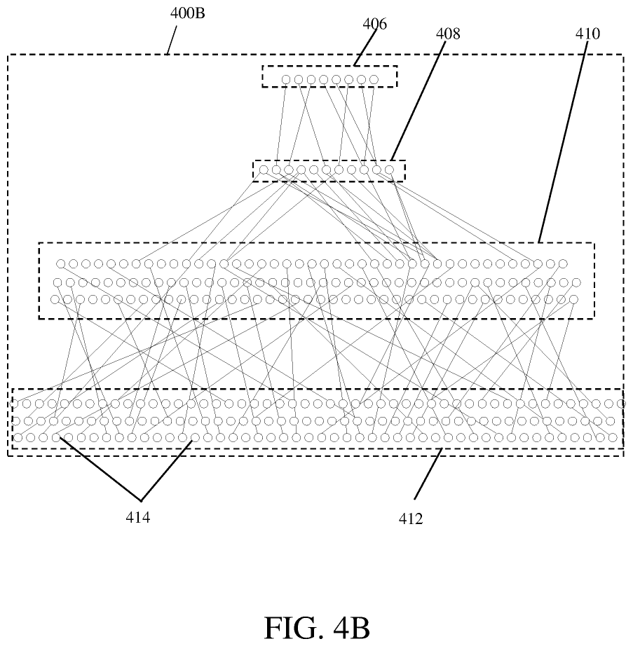

In accordance with an embodiment, the modified GRN is one of: a linear GRN, a staggered GRN and a layered GRN. The modified GRN may also be referred to as a refined GRN. Moreover, the modified GRN is one of the linear GRN, the staggered GRN and the layered GRN. In the layered GRN, different layers exist, for example, the layers of the one or more master regulators are different from the layers of highly regulated or dysregulated genes. An exemplary implementation scenario of the modified GRN is described in detail, for example, in FIG. 4B.

In accordance with an embodiment, the processor 104 is further configured to generate a first GRN for a drug treated person and generate a second GRN for a non-treated person. The processor 104 is further configured to overlay the first GRN over the second GRN to identify alterations at a node level and an edge level and identify one or more highly active edges and one or more inactive edges between the first GRN and the second GRN based on the alterations at the node level and at the edge level. In order to evaluate and understand the alterations between two groups, individual GRNs, such as the first GRN and the second GRN, are generated, overlaid over each other and then, compared at the node and the edge levels. The individual GRNs are generated for different conditions. For example, in an implementation, the first GRN is generated for the drug treated person for a disease cure and the second GRN is generated for the non-treated person. In another implementation, the first GRN is generated for a healthy person and the second GRN is generated for a diseased person. An algorithm, namely. Net-O is used to overlay, analyse and draw mechanistic insights from comparative analysis of two individual GRNs and identify parameters, such as highly active or inactive edges (TF-target interactions) in one condition over another condition.

In accordance with an embodiment, the first GRN is separate from the second GRN. Each of the first GRN and the second GRN is different from each other, for example, control vs disease or drug-treated vs non-treated or healthy vs diseased persons.

In accordance with an embodiment, the processor 104 is further configured to identify one or more of a prognostic biomarker, or a drug response biomarker, or a drug safety biomarker, or a predictive biomarker based on the alterations at the node level and at the edge level. The network overlay approach is further used to identify the potential biomarkers, such as the prognostic biomarker, or the drug response biomarker, or the drug safety biomarker, or the predictive biomarker. The highly dysregulated genes are further investigated and ranked for their potential to be likely biomarkers. The biomarker prioritization matrix considers many factors, such as available literature, association with pathology, accessibility, specificity, etc. to prioritize the biomarkers.

Thus, the system 102 efficiently and reliably identifies the one or more biomarkers associated with the pathology by use of a combination of the GRN, the hierarchical network and the PPI network. The reliable identification of the one or more biomarkers supports development of novel therapeutic strategies. The hierarchical network is based on hierarchy of TFs and targets which makes easy to understand direct and indirect spatial interactions between TFs and targets. Moreover, the hierarchical network includes the identification of master regulators or top TFs (i.e., highly active TFs), and top targets (i.e., highly regulated genes), which is further utilized to generate the modified GRN. The modified GRN provides TF-target interactions not only at the gene level but also, at the protein level. Moreover, the system 102 provides a comprehensive and accurate understanding of gene expression regulation and dysregulation at transcriptional level that takes into account the translational or protein level interactions. In addition to identification of the dysregulated genes, the system 102 is focused on identification of TFs which are involved in gene expression and thereby may be associated with the disease. In addition to prioritization of the TFs and targets, the clusters of genes are identified which may be specifically highlighted for a particular disease. For example, the dysregulation of transcription in cancer makes use of the modified GRN very useful when it comes to cancer diagnostics and novel therapeutics. The system 102 may also be used for discovery of one or more biomarkers, target identification and understanding of a disease mechanism.

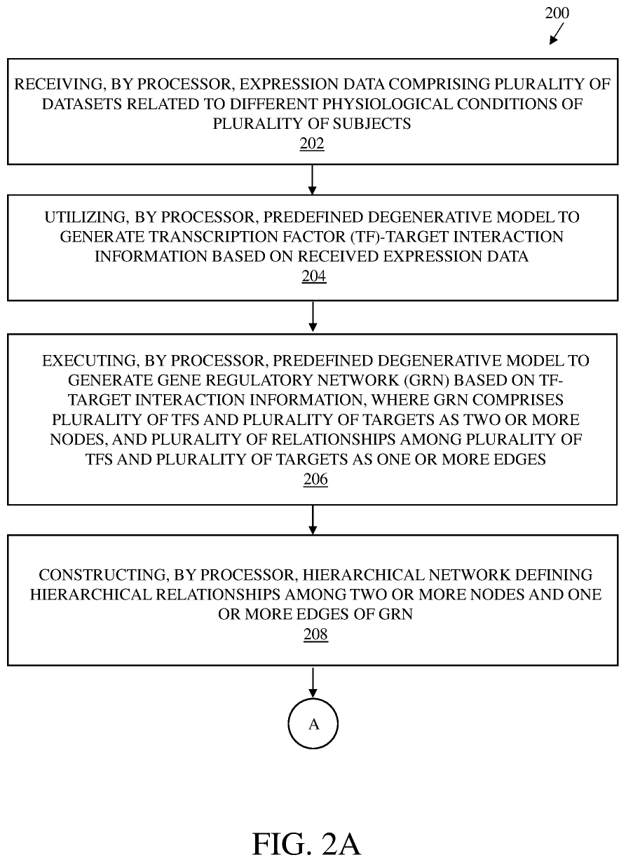

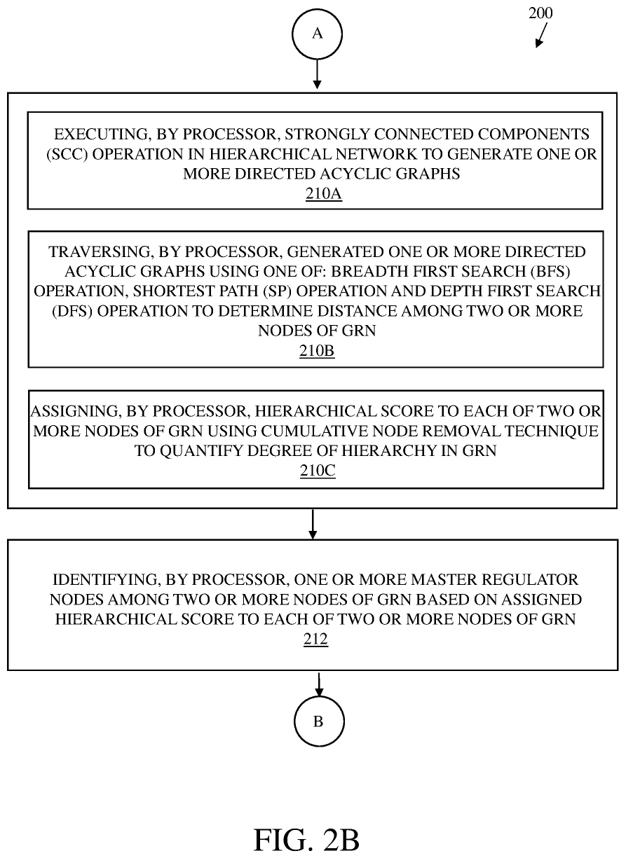

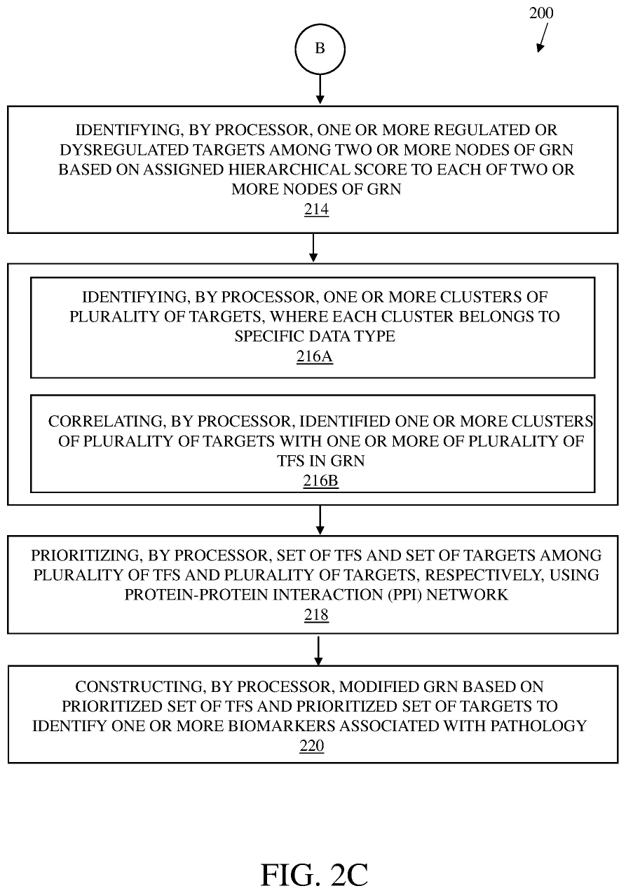

FIGS. 2A to 2C collectively is a flowchart of a method of electronic identification of one or more biomarkers associated with a pathology, in accordance with an embodiment of the present disclosure. FIGS. 2A-2C are described in conjunction with elements from FIG. 1. With reference to FIGS. 2A to 2C, there is shown a flowchart of a method 200 that includes steps 202, 204, 206, 208, 210A, 210B, 210C, 212, 214, 216A, 216B, 218, and 220. The steps 202 to 208 are shown in FIG. 2A, the steps 210A, 210B, 210C, and 212 are shown in FIG. 2B, and the steps 214, 216A, 216B, 218 and 220 are shown in FIG. 2C. The method 200 is executed by the processor 104 of the system 102 (of FIG. 1).

There is provided the method 200 of electronic identification of one or more biomarkers associated with a pathology. The method 200 provides GRN dependent mechanistic insights into pathology and identification of the one or more biomarkers that drive associated transcriptional changes.

At step 202, the method 200 comprises receiving, by the processor 104, the expression data 112 comprising the plurality of datasets related to different physiological conditions of the plurality of subjects. The plurality of datasets corresponds to multiple datasets captured in different physiological conditions of the plurality of patients, such as control vs disease, drug-treated vs non-treated, healthy vs diseased and the like. Moreover, the expression data 112 may include HTS data, micro-array data or scRNA-seq data.

At step 204, the method 200 further comprises utilizing, by the processor 104, the predefined degenerative model 108 to generate the Transcription Factor (TF)-target interaction information based on the received expression data. The predefined degenerative model 108 is used to generate the weighted relationship between TFs and targets based on the expression data 112. The generation of the TF-target interaction information comprises normalization of the plurality of databases, such as hTFtarget database, TcoF-DB v2, TFacts, TF2DNA, ENCODE, and the like, resulting to the normalized database having the small size.

At step 206, the method 200 further comprises executing, by the processor 104, the predefined degenerative model 108 to generate the Gene Regulatory Network (GRN) based on the TF-target interaction information, wherein the GRN comprises the plurality of TFs and the plurality of targets as two or more nodes, and the plurality of relationships among the plurality of TFs and the plurality of targets as one or more edges. The predefined degenerative model 108 is further used to generate the GRN comprising the plurality of TFs and the plurality of targets as nodes and the relationships among the plurality of TFs and the plurality of targets as edges.

At step 208, the method 200 further comprises constructing, by the processor 104, the hierarchical network defining hierarchical relationships among the two or more nodes and the one or more edges of the GRN. After generation of the GRN, the processor 104 is further configured to generate the hierarchical network which defines the hierarchical relationships among the plurality of TFs and the plurality of targets of the GRN.

Now referring to FIG. 2B, at step 210A, the method 200 further comprises executing, by the processor 104, the Strongly Connected Components (SCC) operation in the hierarchical network to generate one or more directed acyclic graphs. The SCC operation is executed in the hierarchical network for generation of the one or more directed acyclic graphs of TFs and targets.

At step 210B, the method 200 further comprises traversing, by the processor 104, the generated one or more directed acyclic graphs using one of: the Breadth First Search (BFS) operation, the Shortest Path (SP) operation and the Depth First Search (DFS) operation to determine distance among the two or more nodes of the GRN. The generated one or more directed acyclic graphs of TFs and targets are traversed using one of the BFS operation, the SP operation and the DFS operation to determine the distance of the nodes from an input gene for which it is required to find stable and deterministic connections.

At step 210C, the method 200 further comprises assigning, by the processor 104, a hierarchical score to each of the two or more nodes of the GRN using a cumulative node removal technique to quantify degree of hierarchy in the GRN. Furthermore, each node of the GRN is assigned the hierarchical score (HS) to quantify the degree of hierarchy in the hierarchical network.

At step 212, the method 200 further comprises identifying, by the processor 104, one or more master regulator nodes among the two or more nodes of the GRN based on the assigned hierarchical score to each of the two or more nodes of the GRN. The one or more master regulator nodes have highest values of the hierarchical score among the two or more nodes of the GRN.

Now referring to FIG. 2C, at step 214, the method 200 further comprises identifying, by the processor 104, one or more regulated or dysregulated targets among the two or more nodes of the GRN based on the assigned hierarchical score to each of the two or more nodes of the GRN. The one or more dysregulated genes are further investigated and ranked for their potential to be likely biomarkers.

At step 216A, the method 200 further comprises identifying, by the processor 104, one or more clusters of the plurality of targets, wherein each cluster belongs to a specific data type.

At step 216B, the method 200 further comprises correlating, by the processor 104, the identified one or more clusters of the plurality of targets with one or more of the plurality of TFs in the GRN.

At step 218, the method 200 further comprises prioritizing, by the processor 104, the set of TFs and the set of targets among the plurality of TFs and the plurality of targets, respectively, using the Protein-Protein Interaction (PPI) network. The PPI network is used to prioritize the set of TFs and the set of targets among the plurality of TFs and the plurality of targets using protein-protein interactions.

At step 220, the method 200 further comprises constructing, by the processor 104, the modified GRN based on the prioritized set of TFs and the prioritized set of targets to identify the one or more biomarkers associated with the pathology. The modified GRN provides more reliable mechanistic interactions between TFs and targets, which further enables the accurate identification of the one or more biomarkers associated with the pathology. The modified GRN is one of the linear GRN, the staggered GRN and the layered GRN.

Moreover, in order to evaluate and understand the alterations between two groups, individual GRNs, such as the first GRN and the second GRN, are generated, overlaid over each other and then, compared at the node and the edge levels. The individual GRNs are generated for different conditions. For example, in an implementation, the first GRN is generated for the drug treated person for a disease cure and the second GRN is generated for the non-treated person.

The steps 202, 204, 206, 208, 210A, 210B, 210C, 212, 214, 216A, 216B 218, and 220 are only illustrative and other alternatives can also be provided where one or more steps are added, one or more steps are removed, or one or more steps are provided in a different sequence without departing from the scope of the claims herein.

FIG. 3A is a diagram of a Gene Regulatory Network (GRN), in accordance with an embodiment of the present disclosure. FIG. 3A is described in conjunction with elements from FIGS. 1 and 2A-2C. With reference to FIG. 3A, there is shown a GRN 300A that comprises a plurality of nodes 302 and a plurality of edges 304. The plurality of nodes 302 represents a plurality of TFs and a plurality of targets and the plurality of edges 304 represents a plurality of relationships among the plurality of TFs and the plurality of targets.

The GRN 300A is generated by the processor 104 of the system 102 (of FIG. 1) on execution of the predefined degenerative model 108. Before execution of the predefined degenerative model 108, the TF-target interaction information is generated based on the expression data 112 received by the processor 104 of the system 102 (of FIG. 1). The TFs-target interaction information may also be referred to as weighted relationships between the plurality of TFs and the plurality of targets.

FIG. 3B is a diagram of a hierarchical network, in accordance with an embodiment of the present disclosure. FIG. 3B is described in conjunction with elements from FIGS. 1, 2A-2C, and 3A. With reference to FIG. 3B, there is shown a hierarchical network 300B that represents hierarchical relationships between the plurality of nodes 302 of the GRN 300A (of FIG. 3A). Each of the weighted relationships between the plurality of TFs and the plurality of targets is used as an input for generation of the hierarchical network 300B. In the hierarchical network 300B, a hierarchical score is assigned to each of the plurality of nodes 302 of the GRN 300A, which is further used to identify one or more master regulators and one or more highly regulated or dysregulated targets (or genes) in the GRN 300A.

FIGS. 4A-4B collectively represent conversion of a random GRN to a layered GRN, in accordance with an embodiment of the present disclosure. FIGS. 4A-4B are described in conjunction with elements from FIGS. 1, 2A-2C, 3A and 3B. With reference to FIG. 4A, there is shown a random GRN 400A comprising a plurality of nodes 402 and a plurality of edges 404. The plurality of nodes 402 represents a plurality of TFs and a plurality of targets and the plurality of edges 404 represents a plurality of relationships among the plurality of nodes 402 of the random GRN 400A. With reference to FIG. 4B, there is shown a layered GRN 400B comprising four layers, such as a first layer 406, a second layer 408, a third layer 410 and a fourth layer 412. There is further shown one or more master regulator nodes 414.

The random GRN 400A is used as an input to construct a hierarchical network (e.g., the hierarchical network 300B, of FIG. 3B). The processor 104 of the system 102 (of FIG. 1) is configured to construct the hierarchical network using the random GRN 400A as the input. In the hierarchical network, a hierarchical score is assigned to each of the plurality of nodes 402 of the random GRN 400A. Based on the assigned hierarchical score, the one or more master regulator nodes 414 are identified among the plurality of nodes 402 of the random GRN 400A. Similarly, on the basis of the assigned hierarchical score, one or more regulated or dysregulated targets (or genes) are identified among the plurality of nodes 402 of the random GRN 400A. After identification of the one or more master regulator nodes 414 and the one or more regulated or dysregulated targets (or genes), the layered GRN 400B is generated. Moreover, in the layered GRN 400B, the first layer 406 represents directly regulated or dysregulated genes. The second layer 408 and the third layer 410, collectively, represent the plurality of nodes 402 (i.e., the plurality of TFs and the plurality of targets) and the fourth layer 412 represent the one or more master regulator nodes 414. In another implementation, there may be more layers in the layered GRN 400B and the arrangement of each of the layers may change.

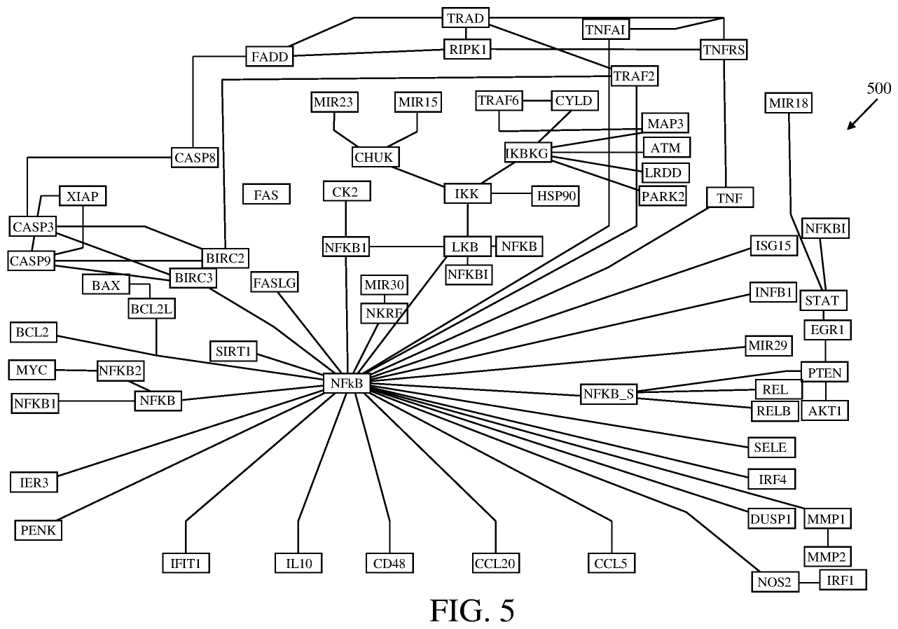

FIG. 5 is a diagram of superimposition of a GRN, a hierarchical network and a Protein-Protein Interaction (PPI) network, in accordance with an embodiment of the present disclosure. FIG. 5 is described in conjunction with elements from FIGS. 1, 2A-2C, 3A-3B, and 4A-4B. With reference to FIG. 5, there is shown a diagram 500 that illustrates superimposition or integration of a GRN (e.g., the GRN 300A, of FIG. 3A), the hierarchical network (e.g., the hierarchical network 300B, of FIG. 3B) and a PPI network. The superimposition of the GRN, the hierarchical network and the PPI network results in generation of a layered GRN (e.g., the layered GRN 400B, of FIG. 4B).

FIG. 6 illustrates a Deep Structural Equation Modelling (DeepSEM model), in accordance with an embodiment of the present disclosure. FIG. 6 is described in conjunction with elements from FIGS. 1, 2A-2C, 3A-3B, 4A-4B, and 5. With reference to FIG. 6, there is shown a DeepSEM model 600 that includes an input data 602, an encoder 604 comprising a multi-layer perceptron (MLP) neural network 604A and a GRN layer 604B, a decoder 606 and an output data 608.

The input data 602 includes either a micro-array data or scRNA-seq data as gene expression data along with an internal database. The MLP neural network 604A is a feed forward artificial neural network that generates a set of outputs from a set of inputs. The set of inputs corresponds to the input data 602. Furthermore, at the GRN layer 604B, the GRN (e.g., the GRN 300A of FIG. 3A) is constructed using shared weights of the encoder 604 and the decoder 606. The shared weights merely represent the connections between TFs and targets. The absolute value of each element of the shared weights is used to rank the possibility of the regulatory relationships between genes. The output data 608 includes prioritized set of TFs and targets including edge weights too.

In the DeepSEM model 600, the weights of both the encoder 604 and the decoder 606 represent adjacency matrix of the GRN 300A. The DeepSEM model 600 does not require any additional experimental data, such as open chromatin information, CHIP sequencing (CHIP-seq) data or TF binding motifs to infer the GRN 300A. Additionally, by explicitly modelling the GRN 300A, the DeepSEM model 600 is more transparent than a conventional neural network model. Moreover, the DeepSEM model 600 may reduce the overfitting problem of conventional deep learning models by greatly restricting the parameter space. The Variational Auto-Encoder (VAE) of the DeepSEM model 600 contains four modules: an encoder, a GRN layer, an inverse GRN layer and a decoder. The encoder 604 and the decoder 606 are both MLPs considering one gene as an input and the weights of the encoder 604 and the decoder 606 are shared between different genes. The GRN layer and the inverse GRN layer are both gene interaction matrices, which explicitly model the GRN 300A and guide the information flow of the neural network (i.e., the MLP neural network 604A). The DeepSEM model 600 is used to reduce false positive data and to generate the TFs-target interaction information. Moreover, the performance of the DeepSEM model 600 can further be enhanced by replacing linear layers by convolutional layers in the encoder 604 and the decoder 606, which can extract more features from the input data 602.

FIG. 7 is a flowchart that describes how hierarchical scores are assigned to each of TFs and targets in a hierarchical network, in accordance with an embodiment of the present disclosure. FIG. 7 is described in conjunction with elements from FIGS. 1, 2A-2C. 3A-3B. 4A-4B, 5, and 6. With reference to FIG. 7, there is shown a flowchart 700 that includes a series of operations 702 to 726.

At operation 702, a GRN network (e.g., the GRN 300A) of a plurality of TFs and a plurality of targets is used as an input.

At operation 704, a hierarchical network (e.g., the hierarchical network 300B) is generated using the GRN network as the input.

At operation 706, the SCC operation is executed in the hierarchical network to generate clusters of TFs and targets, where each cluster of TF-target has similar properties. The execution of the SCC operation reduces complexity of the hierarchical network.

At operation 708, after execution of the SCC operation, a directed graph (may also be represented as G) is generated, which have a plurality of relationships directed from TFs to genes.

At operation 710, after execution of the SCC operation, another directed graph (may also be represented as GT) is generated, which have a plurality of relationships directed from genes to TFs.

At operation 712, each of the directed graph (i.e., G) and the other-directed graph (i.e., GT) is traversed using a graph traversal technique, such as the Breadth First Search, the Shortest Path or the Depth First Search algorithms, to determine shortest distance between two nodes.

At operation 714, after traversing each of the directed graph (i.e., G) and the other-directed graph (i.e., GT), a hierarchical tree comprising G levels of TFs-targets, the one or more master regulator nodes, and highly regulated/dysregulated genes, is generated.

At operation 716, after traversing each of the directed graph (i.e., G) and the other-directed graph (i.e., GT), another hierarchical tree comprising GT levels of TFs-targets, the one or more master regulator nodes, and highly regulated/dysregulated genes, is generated.

At operation 718, the generated other hierarchical tree having GT levels is reversed. After reversal, the generated other hierarchical tree having GT levels has the plurality of relationships directed from TFs to genes.

At operation 720, the cumulative node removal technique is used. The hierarchical tree comprising G levels of TFs-targets and the other hierarchical tree comprising GT levels of TFs-targets have some similarities and differences as well. The differences between the hierarchical tree comprising G levels of TFs-targets and the other hierarchical tree comprising GT levels of TFs-targets are identified by use of the cumulative node removal technique. For example, after removal of a few nodes from the other hierarchical tree comprising GT levels of TFs-targets, the hierarchical tree comprising G levels of TFs-targets and the other hierarchical tree comprising GT levels of TFs-targets become similar then, the removed nodes have significance. Alternatively stated, in order to identify the significant TFs between the hierarchical tree comprising G levels of TFs-targets and the other hierarchical tree comprising GT levels of TFs-targets, the cumulative node removal technique is used.

At operation 722, each of the hierarchical tree comprising G levels of TFs-targets and the other hierarchical tree comprising GT levels of TFs-targets is compared with each other to generate hierarchical score in the hierarchical tree comprising G levels.

At operation 724, each of the hierarchical tree comprising G levels of TFs-targets and the other hierarchical tree comprising GT levels of TFs-targets is compared with each other to generate hierarchical score in the other hierarchical tree comprising GT levels.

At operation 726, a tabular representation of the hierarchical scores assigned to TFs and targets is generated.

There are few use-cases of the approach used in the flowchart 700. A first use case is when any Differential Expressed Genes (DEG) is given in a specific indication, a set of TFs can be prioritized by assigning a hierarchical score. A second use case is when any DEG is given is a specific indication, the one or more master regulator nodes can be identified along with the regulation path. A third use case is when any DEG is given is a specific indication, the one or more activated or repressing master regulator nodes can be identified along with the regulation path.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

1. A system for electronic identification of one or more biomarkers associated with a pathology, comprising:
a processor configured to:
receive an expression data comprising a plurality of datasets related to different physiological conditions of a plurality of subjects;
utilize a predefined degenerative model to generate a Transcription Factor (TF)-target interaction information based on the received expression data;
execute the predefined degenerative model to generate a Gene Regulatory Network (GRN) based on the TF-target interaction information, wherein the GRN comprises a plurality of TFs and a plurality of targets as two or more nodes, and a plurality of relationships among the plurality of TFs and the plurality of targets as one or more edges;
construct a hierarchical network defining hierarchical relationships among the two or more nodes and the one or more edges of the GRN;
prioritize a set of TFs and a set of targets among the plurality of TFs and the plurality of targets, respectively, using a Protein-Protein Interaction (PPI) network; and
construct a modified GRN based on the prioritized set of TFs and the prioritized set of targets to identify the one or more biomarkers associated with the pathology.

2. The system according to claim 1, wherein the processor is further configured to:
execute a Strongly Connected Components (SCC) operation in the hierarchical network to generate one or more directed acyclic graphs;
traverse the generated one or more directed acyclic graphs using one of: a Breadth First Search (BFS) operation, a Shortest Path (SP) operation and a Depth First Search (DFS) operation to determine distance among the two or more nodes of the GRN; and
assign a hierarchical score to each of the two or more nodes of the GRN using a cumulative node removal technique to quantify a degree of hierarchy in the GRN.

3. The system according to claim 2, wherein the processor is further configured to:
identify one or more master regulator nodes among the two or more nodes of the GRN based on the assigned hierarchical score to each of the two or more nodes of the GRN.

4. The system according to claim 2, wherein the processor is further configured to:
identify one or more regulated or dysregulated targets among the two or more nodes of the GRN based on the assigned hierarchical score to each of the two or more nodes of the GRN.

5. The system according to claim 1, wherein the processor is further configured to:
identify one or more clusters of the plurality of targets, wherein each cluster belongs to a specific data type; and
correlate the identified one or more clusters of the plurality of targets with one or more of the plurality of TFs in the GRN.

6. The system according to claim 1, wherein the generation of the TF-target interaction information comprises normalization of a plurality of databases resulting to a normalized database.

7. The system according to claim 1, wherein the predefined degenerative model is an autoencoder model.

8. The system according to claim 1, wherein the expression data comprises either high sequencing throughput (HTS) data, or a micro-array data or a single cell sorting, RNA extraction, reverse transcription, amplification, library construction, sequencing and subsequent bioinformatic analysis (scRNA-seq) data.

9. The system according to claim 1, wherein the modified GRN is one of: a linear GRN, a staggered GRN and a layered GRN.

10. The system according to claim 1, wherein the processor is further configured to:
generate a first GRN for a drug treated person;
generate a second GRN for a non-treated person;
overlay the first GRN over the second GRN to identify alterations at a node level and an edge level; and
identify one or more highly active edges and one or more inactive edges between the first GRN and the second GRN based on the alterations at the node level and at the edge level.

11. The system according to claim 10, wherein the first GRN is separate from the second GRN.

12. The system according to claim 10, wherein the processor is further configured to identify one or more of: a prognostic biomarker, or a drug response biomarker, or a drug safety biomarker, or a predictive biomarker based on the alterations at the node level and at the edge level.

13. A method of electronic identification of one or more biomarkers associated with a pathology, comprises:
receiving, by a processor, an expression data comprising a plurality of datasets related to different physiological conditions of a plurality of subjects;
utilizing, by the processor, a predefined degenerative model to generate a Transcription Factor (TF)-target interaction information based on the received expression data;
executing, by the processor, the predefined degenerative model to generate a Gene Regulatory Network (GRN) based on the TF-target interaction information, wherein the GRN comprises a plurality of TFs and a plurality of targets as two or more nodes, and a plurality of relationships among the plurality of TFs and the plurality of targets as one or more edges;
constructing, by the processor, a hierarchical network defining hierarchical relationships among the two or more nodes and the one or more edges of the GRN;
prioritizing, by the processor, a set of TFs and a set of targets among the plurality of TFs and the plurality of targets, respectively, using a Protein-Protein Interaction (PPI) network; and
constructing, by the processor, a modified GRN based on the prioritized set of TFs and the prioritized set of targets to identify the one or more biomarkers associated with the pathology.

14. The method according to claim 13, wherein the method further comprises:
executing, by the processor, a Strongly Connected Components (SCC) operation in the hierarchical network to generate one or more directed acyclic graphs;
traversing, by the processor, the generated one or more directed acyclic graphs using one of: a Breadth First Search (BFS) operation, a Shortest Path (SP) operation and a Depth First Search (DFS) operation to determine distance among the two or more nodes of the GRN; and
assigning, by the processor, a hierarchical score to each of the two or more nodes of the GRN using a cumulative node removal technique to quantify degree of hierarchy in the GRN.

15. The method according to claim 14, wherein the method further comprises:
identifying, by the processor, one or more master regulator nodes among the two or more nodes of the GRN based on the assigned hierarchical score to each of the two or more nodes of the GRN.

16. The method according to claim 14, wherein the method further comprises:
identifying, by the processor, one or more regulated or dysregulated targets among the two or more nodes of the GRN based on the assigned hierarchical score to each of the two or more nodes of the GRN.

17. The method according to claim 13, wherein the method further comprises:
identifying, by the processor, one or more clusters of the plurality of targets, wherein each cluster belongs to a specific data type; and
correlating, by the processor, the identified one or more clusters of the plurality of targets with one or more of the plurality of TFs in the GRN.

18. The method according to claim 13, wherein the method further comprises:
generating, by the processor, a first GRN for a drug treated person;
generating, by the processor, a second GRN for a non-treated person;
overlaying, by the processor, the first GRN over the second GRN to identify alterations at a node level and an edge level; and
identifying, by the processor, one or more highly active edges and one or more inactive edges between the first GRN and the second GRN based on the alterations at the node level and the edge level.

19. The method according to claim 18, wherein the first GRN is separate from the second GRN.

20. The method according to claim 18, wherein the method further comprises identifying, by the processor, one or more of: a prognostic biomarker, or a drug response biomarker, or a drug safety biomarker, or a predictive biomarker based on the alterations at the node level and at the edge level.
